(19) United States
(12) Patent Application Publication
(10) Pub. No.: US 2010/0087850 A1
(43) Pub. Date: Apr. 8, 2010

(54) Mechanical Embolectomy Device and Method

(76) Inventor: Nasser Razack, Bradenton, FL (US)

Correspondence Address: Maxey Law Offices, PLLC, 13630 58TH ST. NORTH, SUITE 101, CLEARWATER, FL, 33760, US

(21) Appl. No.: 12/244,791

(22) Filed: Oct. 3, 2008

(51) Int. Cl. A61M 29/00

(52) U.S. Cl. 606/200

(57) ABSTRACT

An embolectomy device includes an elongated shaft positionable in and movable within a catheter. The elongated shaft having a distal end portion. An expander portion having proximal and distal ends. The expander portion having a first plurality of legs connecting the proximal end of the expander portion to the distal end portion of the elongated shaft and a second plurality of legs connecting the distal end of the expander portion to the proximal end of the intermediate elongated shaft. The expander portion being biased to expand laterally outward from the long axis of the embolectomy device when the expander portion has been moved out of the catheter to engage a clot. A retrieval portion can be provided proximally of the expander portion. A retrieval net can be fixed to the retrieval portion. The expander portion can be at least partially covered by a net. A method for removing clots and other occlusions for body canals is also provided.

### FILED OF THE INVENTION

The present invention relates generally to embolectomy, and more particularly, relating to a mechanical embolectomy device including a clot expander and a clot particle retriever, and method of using the same.

### BACKGROUND OF THE INVENTION

There are currently in excess of 700,000 new or recurrent strokes every year in the United States. It is the third leading cause of death in the United States after coronary artery disease and cancer. Approximately 40% (>250,000) of strokes are due to large vessel occlusion, potentially requiring a device for treatment.

Treatment must begin with an evaluation of the patient. Diagnostic neuroimaging is used to obtain noninvasive real-time information about the patient. The goal of therapy is to restore perfusion to the ischemic but potentially salvageable brain tissue rather than to the irreversibly damaged brain tissue, since re-establishing blood flow to such damaged tissue can cause complications such as hemorrhage. Determining the cause and location of the blockage is critical to planning the treatment approach. The most common cause of ischemic stroke is acute embolic occlusion. Most patients with acute ischemic stroke have thromboembolic material occluding large cerebral vessels and hence disruption of cerebral blood flow. Removal of the arterial occlusion in a timely manner can provide a substantial reduction in the size and severity of the cerebral infarction, and improvement in the level of disability among survivors. Treatment varies depending on whether the lesion is proximal or distal, whether there is underlying atherosclerotic stenosis at the occlusion site, and whether the proximal extracranial vessel is opened or closed. Where there are proximal arterial occlusions, the physician may attempt clot retrieval, supplemented by direct catheter-directed thrombolysis. Severe stenosis proximal to the occlusion will usually require treatment of the stenosois before or immediately after restoring intracranial flow.

A current treatment for acute ischemic stroke is intravenous thrombolysis using tissue-type plasminogen activator (TPA). TPA is a naturally occurring enzyme that activates plasminogen into active plasmin, which dissolves fibrin. The dissolution of fibrin in a clot causes thrombolysis. This treatment is suitable for smaller clots, but has limited utility for patients with large clots, such as are often present in acute occlusions of the internal carotid artery (ICA), proximal middle cerebral artery (MCA), and basilar artery (BA). Also, TPA therapy has significant time constraints, and is generally effective only if given within 3-6 hours of stroke symptom onset. Contraindications to TPA and these time constraints led to mechanical embolectomy.

Mechanical removal of the thrombus is the goal of mechanical embolectomy. Lytic therapy is necessary for non-accessible locations. Mechanical embolectomy is the process by which a mechanical device is inserted into the body, moved through the affected body canal to the site of the occlusion, and then used to mechanically remove the occlusion from the canal to restore blood flow. One such device is the Mechanical Embolus Removal in Cerebral Ischemia (Merci) retrieval device (Concentric Medical, Mountain View, Calif.), which is currently available for routine clinical use in acute ischemic stroke within 9 hours of onset. This device is a flexible and tapered nickel titanium wire with a helically shaped distal tip that can be deployed intra-arterially to entrap and retrieve large vessel intracerebral clots. Other devices are in various stages of development.

### SUMMARY OF THE INVENTION

In general, in one aspect, an embolectomy device is provided. The embolectomy device includes an elongated shaft positionable in and movable within a catheter. The elongated shaft having a distal end portion. An intermediate elongated shaft positionable in an movable with the catheter. The intermediate elongated shaft having proximal and distal ends. An expander portion having proximal and distal ends. The expander portion having a first plurality of legs connecting the proximal end of the expander portion to the distal end portion of the elongated shaft and a second plurality of legs connecting the distal end of the expander portion to the proximal end of the intermediate elongated shaft. A retrieval portion having a plurality of legs having proximal and distal portions. The proximal portion being attached to the distal end of the intermediate elongated shaft at the proximal portion of the of plurality of legs of the retrieval portion. The plurality of legs of the retrieval portion being movable laterally outward from a long axis of embolectomy device when the retrieval portion has been moved out of the catheter. The expander portion being biased to expand laterally outward from the long axis of the embolectomy device when the expander portion has been moved out of the catheter to engage a clot.

An atraumatic distal portion can extend distally from the distal end portion of the plurality of legs of the of retrieval portion. A retrieval net can be fixed to the retrieval portion. The net can be constructed from a polymeric material. The expander portion can be at least partially covered by a net. The net can comprises a biocompatible or non-thrombegenic material. The embolectomy device can further include the catheter.

In general, in another aspect, an embolectomy device is provided. The embolectomy device includes an elongated shaft positionable in and movable within a catheter. The elongated shaft having a distal end portion. An intermediate elongated shaft positionable in an movable with the catheter. The intermediate elongated shaft having proximal and distal ends. An expander portion having proximal and distal ends, and having a first plurality of legs connecting the proximal end of the expander portion to the distal end portion of the elongated shaft and a second plurality of legs connecting the distal end of the expander portion to the proximal end of the intermediate elongated shaft. A retrieval portion having a plurality of legs having proximal and distal ends and a mid-portion between the proximal and distal ends, and the proximal end being attached to the distal end of the intermediate elongated shaft at the proximal end of the of plurality of legs of the retrieval portion. The mid-portion of the legs being movable laterally outward from a long axis of the embolectomy device when the retrieval portion has been moved out of the catheter. The expander portion being biased to expand laterally outward from the long axis of the embolectomy device when the expander portion has been moved out of the catheter to engage a clot.

A retrieval net can be fixed to the plurality of legs of the retrieval portion between the mid-portion and the distal end thereof. The plurality of legs of the retrieval portion can be spring arms. The spring arms can comprise a bend at the mid-portion defining proximal and distal portions, the bend biasing the spring arms to a lateral outward position. An atraumatic distal portion can extend distally from the distal end portion of the plurality of legs of the of retrieval portion. The expander portion can be at least partially covered by a net. The net can comprises a biocompatible or non-thrombegenic material. The embolectomy device can further include the catheter. The spring arms can be integral with the intermediate elongated shaft. The spring arms can be attached to the intermediate elongated shaft.

In general, in another aspect, an embolectomy device is provided. The embolectomy device includes an elongated shaft positionable in and movable within a catheter. The elongated shaft having a distal end portion. An expander portion having proximal and distal ends. The expander portion having a first plurality of legs connecting the proximal end of the expander portion to the distal end portion of the elongated shaft and a second plurality of legs connecting the distal end of the expander portion to the proximal end of the intermediate elongated shaft. The expander portion being biased to expand laterally outward from the long axis of the embolectomy device when the expander portion has been moved out of the catheter to engage a clot.

An atraumatic distal portion can extend distally from the distal end of the expander portion. The expander portion can be at least partially covered by a net. The net can comprises a biocompatible or non-thrombegenic material. The embolectomy device can further include the catheter.

In general, in another aspect, a method of performing an embolectomy is provided. The method includes the steps of providing an embolectomy device comprising an elongated shaft positionable in and movable within a catheter, the elongated shaft having a distal end portion, an intermediate elongated shaft positionable in an movable with the catheter, the intermediate elongated shaft having proximal and distal ends, an expander portion having proximal and distal ends, the expander portion having a first plurality of legs connecting the proximal end of the expander portion to the distal end portion of the elongated shaft and a second plurality of legs connecting the distal end of the expander portion to the proximal end of the intermediate elongated shaft, a retrieval portion having a plurality of legs having proximal and distal portions, the proximal portion being attached to the distal end of the intermediate elongated shaft at the proximal portion of the of plurality of legs of the retrieval portion, the plurality of legs of the retrieval portion being movable laterally outward from a long axis of the elongated shaft when the retrieval portion has been moved out of the catheter, the expander portion being biased to expand laterally outward from the long axis of the embolectomy device when the expander portion has been moved out of the catheter to engage an occlusion, and a retrieval net fixed to the retrieval portion. The embolectomy device is manipulated within a catheter to position the expander portion across an occlusion in a body canal. The expander portion is expanded to move the occlusion toward the body canal, thereby creating a lumen. The expander portion is retracted. The retrieval portion is moved proximally so as to engage the occlusion with the retrieval net. The catheter and embolectomy device are removed from the body canal as one with portions of the occlusion contained by the catheter and retrieval portion of the embolectomy device.

In general, in another aspect, a method of performing an embolectomy is provided. The method includes the steps of providing an embolectomy device including an elongated shaft positionable in and movable within a catheter, the elongated shaft having a distal end portion, an expander portion having proximal and distal ends, the expander portion having a first plurality of legs connecting the proximal end of the expander portion to the distal end portion of the elongated shaft and a second plurality of legs connecting the distal end of the expander portion to the proximal end of the intermediate elongated shaft, the expander portion being biased to expand laterally outward from the long axis of the embolectomy device when the expander portion has been moved out of the catheter to engage an occlusion. The embolectomy device is manipulated within a catheter to position the expander portion across an occlusion in a body canal. The expander portion is expanded to move the occlusion toward the body canal, thereby creating a lumen. The expander portion is retracted, and the catheter and embolectomy device are removed from the body canal leaving the lumen open.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is a side elevation of an embolectomy device in accordance with the principles of the present invention shown fully extended from the microcatheter;

FIGS. 2-5 are side elevation views of the embolectomy device in accordance with the principles of the present invention shown partially extended from the microcatheter in various stages;

FIGS. 6-11 are side elevation views of the embolectomy device in accordance with the principles of the present invention shown at various stages of operation removing a clot from a body canal;

FIG. 12 is a side elevation of an alternative embodiment;

FIG. 13 is a side elevation of an alternative embodiment; and

FIGS. 14-17 are side elevation views of the embolectomy device of FIG. 13 at various stages of operation creating a lumen through in a clot in a body canal

### DETAILED DESCRIPTION OF THE INVENTION

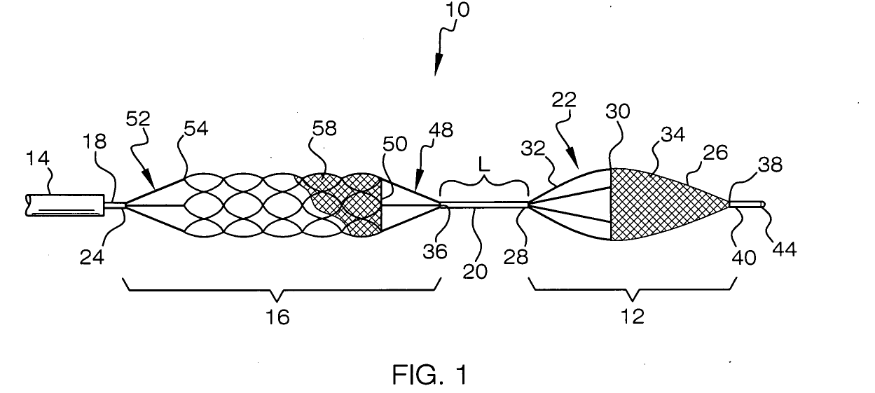

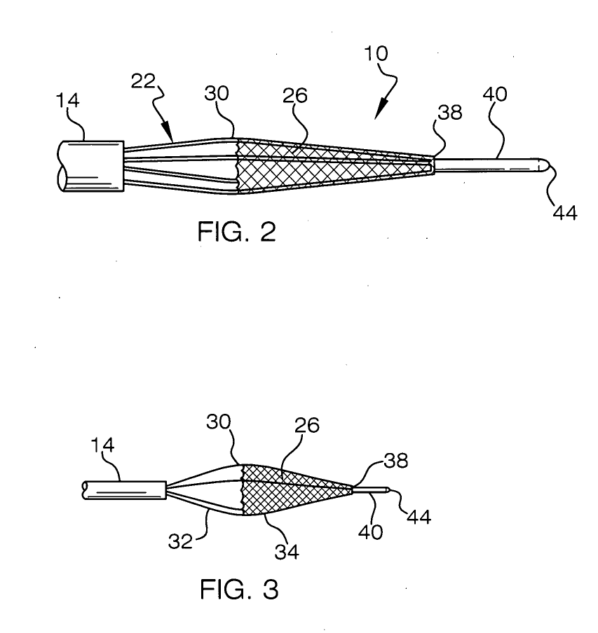

There is shown in FIGS. 1-5 an embolectomy device 10 according to the invention. In FIG. 1, the embolectomy device 10 is shown fully extended. In FIGS. 2-5, the embolectomy device 10 is shown partially extended in various stages. The embolectomy device 10 is positionable in and movable within a catheter 14, such as a microcatheter. The embolectomy device 10 includes an elongated shaft such as proximal wire 18 extending through an opening in the microcatheter 14, an expander portion 16, and a retrieval portion 12. The proximal wire 18 can have a proximal end (not shown) and distal end 24. The expander portion 16 can be provided at the distal end 24 of the proximal wire 18. The retrieval portion 12 can be provided distally at a spaced distance from the expander portion 16, and can be connected to the expander portion by an intermediate elongated shaft or intermediate connecting wire 20.

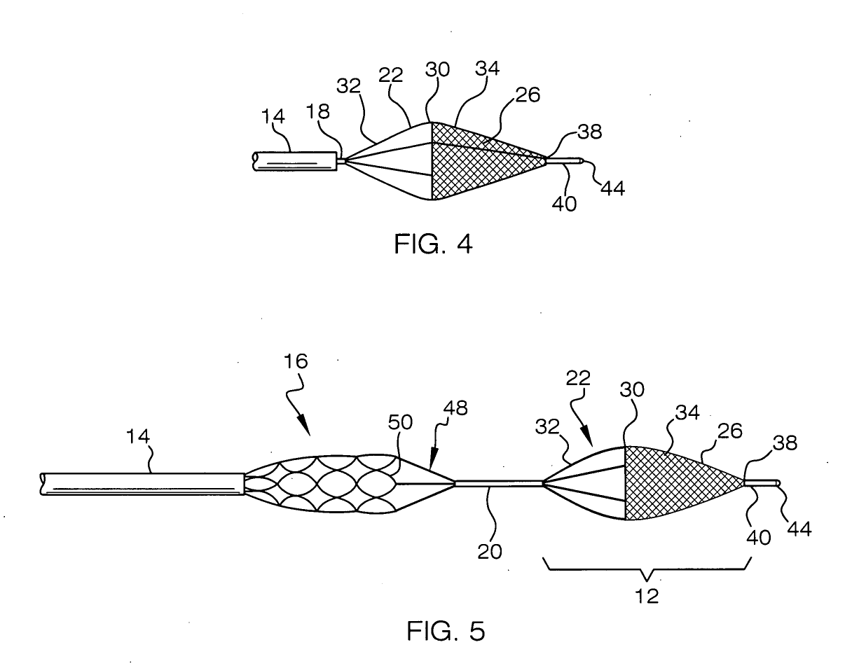

The retrieval portion 12 may be formed from or attached to the intermediate connecting wire 20. The retrieval portion 12 can have a plurality of legs such as spring arms 22 that are connected to the intermediate connecting wire 20. The spring arms 22 are disposed about a long axis A of the embolectomy device 10. The spring arms 22 are biased or otherwise moveable to extend partially outwardly (FIG. 3) when not constrained entirely by the microcatheter 14. When unconstrained by the microcatheter 14 (FIG. 4) the spring arms 22 can extend laterally outward a maximum distance. Movement of the spring arms 22 relative to the microcatheter 14 can thereby be used to control the lateral extension of the spring arms 22 within a range of possible distances between full extension and full retraction.

A retrieval net 26 can be provided on the spring arms 22 to assist in engaging and removing a clot or other obstruction from a body canal. The net 26 can be constructed of a flexible, thin-walled material such that when the spring arms 22 are positioned within the microcatheter 14, the net can also be contained within the microcatheter 14. The net 26 can also move freely into and out of the microcatheter 14 with the retrieval portion 12. The net 26 can be constructed from a porous or a substantially non-porous material, such as a flexible plastic net or solid sheet material, and/or a biocompatible or non-thrombogenic polymer. The net 26 can be formed from a polymeric material that is adhered or otherwise securely fixed to the spring arms

The precise arrangement and construction of the spring arms 22 can be varied. In the embodiment shown, the spring arms 22 are elongated and substantially radially disposed about the long axis A of the embolectomy device 10. The spring arms 22 can be formed separately and attached to the intermediate guide wire 20, or the spring arms 22 can be integral with the intermediate guide wire 20 and formed by suitable techniques, such as, but not limited to computer-controlled laser cutting, plastic injection molding, or casting. The spring arms 22 can have a mid-portion 30, which can have a bend, crimp, curve, or other biasing or moving feature or structure which causes the legs/spring arms 22 to extend laterally outward relative to the long axis A of the embolectomy device 10. In one embodiment, the spring arms 22 are at least partially made of an elastic material, such as plastic or metal. The mid-portion 30 defines proximal portions 32 and distal portions 34 of the spring arms 22. The retrieval portion 12 can be attached to a distal end 28 of the intermediate connecting wire 20 at the proximal portion 32 of the spring legs 22. When the proximal portions 32 are positioned within the microcatheter 14, the proximal portions 32 are retained in a laterally inward position by the inside wall of the microcatheter (FIG. 2). As the embolectomy device 10 is extended from the microcatheter 14, the spring arms 22 are permitted under the influence of the biasing to extend laterally outward (FIG. 3). As the embolectomy device 10 is moved further out of the microcatheter 14, the spring arms 22 are moved by the biasing completely out of the microcatheter 14 and extend a maximum lateral distance from the long axis A of the embolectomy device 10 (FIG. 4). The distal portions 34 extend from a lateral maximum distance at the bend 30 to a lateral minimum distance at the distal end 38 of the spring arms 22. The lateral outward extension of the legs/spring arms 22 can thereby be controlled by the distance which the proximal portions 32 are moved out of the microcatheter 14.

The net 26 is joined to the distal portions 34 of the spring arms 22, such that when the spring arms 22 are laterally extended, the net 26 is opened to that in one embodiment is a substantially conical configuration and retained in that position by the spring arms 22, as shown in FIG. 4. In this position, the net 26 can engage, capture and retain a clot or other obstruction during an embolectomy. The net 26 also retains debris which otherwise might be left in the body canal. An atraumatic distal portion 40 can be provided at the distal end of the embolectomy device 10 for preventing the embolectomy device from penetrating or piercing a body canal as it is moved therethrough. The distal portion 40 can be part of the intermediate connecting wire 20 or can be a separate structure to which the spring arms 22 are affixed. The distal portion 40 can be flexible and non-rigid. The distal portion 40 can include a blunt atraumatic tip 44.

The expander portion 16 may be formed from or attached to the proximal wire 18. The expander portion 16 is disposed about the long axis A of the embolectomy device 10, and may be biased or otherwise moveable to extend outwardly when not constrained by the microcatheter 14. The expander portion 16 may have a plurality of legs 48 that connect a distal end 50 of the expander portion to a proximal end 36 of the intermediate connecting wire 20, and a plurality of legs 52 that connect a proximal end 54 of the expander portion to the distal end 24 of the proximal wire 18. Legs 48 and 52 may be spring arms, and biased or otherwise moveable to extend outwardly (FIG. 5) when not constrained by the microcatheter 14, similarly to that of spring arms 22. Additionally, as with spring arms 22, the construction and arrangement of spring arms 48 and 52 can be varied. In the embodiment shown, the spring arms 48 and 52 are elongated and substantially radially disposed about a long axis A of the embolectomy device 10. Spring arms 48 and 52 can be formed separately and attached to the proximal wire 18 and intermediate connecting wire 20, respectively. Spring arms 48 and 52 can be integral with the proximal wire 18 and intermediate connecting wire 20, respectively, and can be formed by suitable techniques, such as, but not limited to, computer-controlled laser cutting, plastic injection molding, or casting. In one embodiment, spring arms 48 and 52 are at least partially made of an elastic material, such as plastic or metal.

A mesh or net 58 can cover a portion or all of the expander portion 16 in order to better engage clot material. The net 58 can be constructed from a porous or a substantially non-porous material, such as a flexible plastic net or solid sheet material, and/or a biocompatible or non-thrombogenic polymer. The net 58 can be integral with the expander portion. Expansion of the expander portion 16 is controlled by suitable means, such as the presence of the microcatheter 14. When the microcatheter 14 is removed or pulled back from the expander portion 16, the expander is expanded either by a spring force or a suitable motor.

The expander portion 16 can be of different designs, but can be a mesh metallic or polymeric device similar to a stent in that it is capable of a first position in which it is tightly compacted laterally, and in another position expands laterally. It is important to understand the expander portion 16 is only stent like, and is not intended to be left in place within a body canal. The expander portion 16 uses this lateral expansion force to spread clot material concentrically outwardly toward the walls of the body canal, opening a lumen through the body canal. In the high energy, compacted state the expander portion 16 is contained within a catheter. When moved out of the catheter, the expander portion 16 assumes an expanded, lower energy state. The expander portion 16 can be completely or partially coated with a polymer, and/or with a therapeutic substance. The expander portion 16 can be porous, non-porous, or partially porous. The expander portion can be formed by suitable techniques, such as, but not limited to, computer-controlled laser cutting, plastic injection molding, or casting.

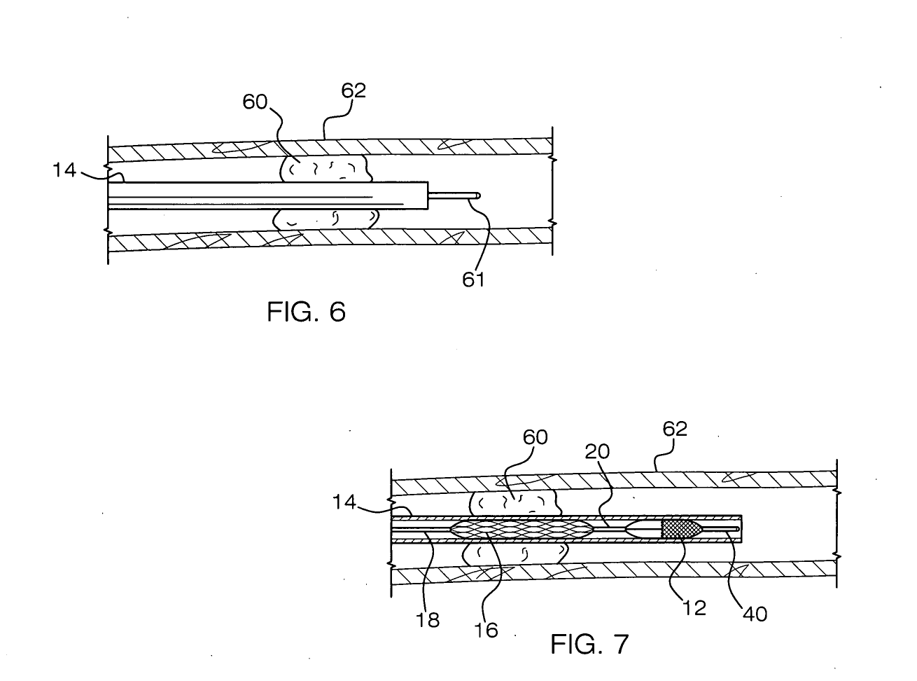

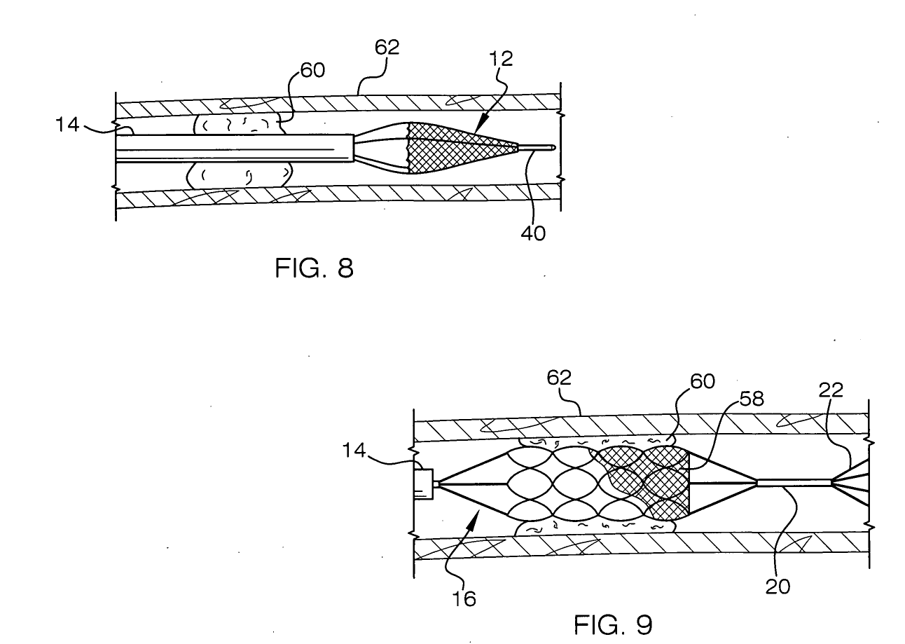

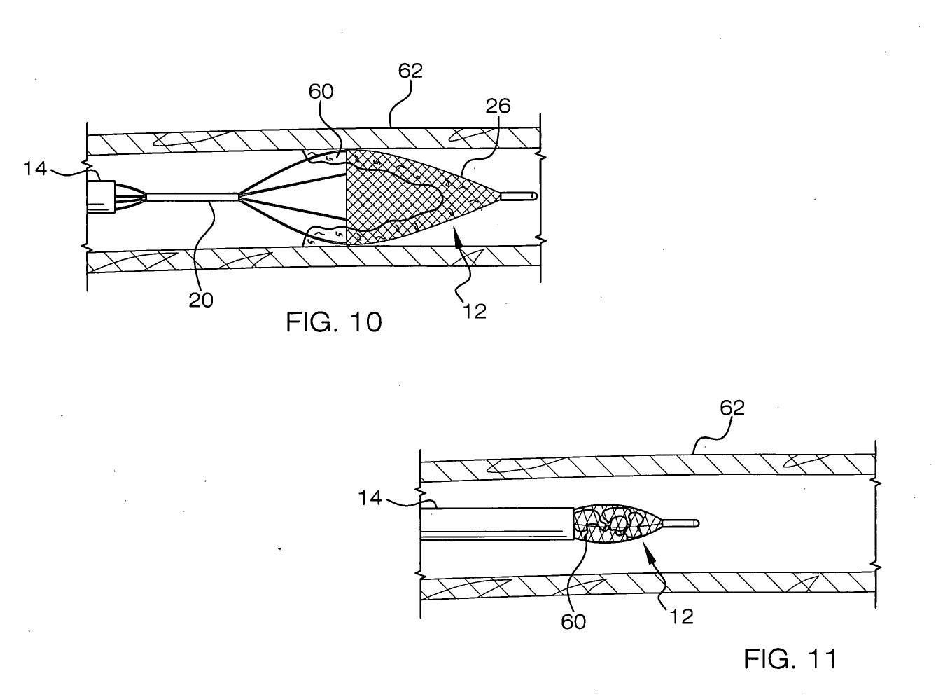

Operation of the embolectomy device 10 is shown in FIGS. 6-11. In FIG. 6, the microcatheter 14 shown positioned within the body canal 62 through and beyond clot 60 using a steerable microwire 61 and standard interventional radiology techniques. With the microcatheter 14 in position in the body canal 62 and through the clot 60, the steerable microwire is removed from the microcatheter. A contrasting agent can be injected into the microcatheter 14 to confirm the microcatheter is correctly positioned through the clot 60. While maintaining the position of microcatheter 14 the embolectomy device 10 is inserted into the microcatheter to position the expander portion 16 across the clot 60 (FIG. 7). The embolectomy device 10 is extended from the microcatheter 14 by withdrawing the microcatheter 14 proximally while maintaining the positioning of the embolectomy device. As the proximal portions 32 of the retrieval portion 16 emerge from the microcatheter 14 (FIG. 8), the spring arms 22 begin to extend laterally outward, which extends the mid-portions 30 laterally outward. As the spring arms 22 are moved completely out of the microcatheter 14 (FIG. 9 and 10), the spring arms 22 extend outward a maximum lateral distance relative to the long axis A of the embolectomy device 10. Continued withdrawal of the microcatheter 14 exposes the expander portion 16, which is expanded by either spring force or a suitable motor, pressing the clot 60 against the body canal 62 (FIG. 9) and creating a lumen. The expander portion 16 can then be collapsed and retracted into the microcatheter 14 by moving the microcatheter distally over the expander portion. With the expander portion 16 contained within the microcatheter 14, the embolectomy device 10 can be further retracted, by either moving the microcatheter distally over the embolectomy device 10, moving the embolectomy device proximally into the microcatheter, or a combination of both such that clot 60 will be engaged by the net 26 (FIG. 10) of the retrieval portion 12 and at least partially pulled into the microcatheter (FIG. 11). The microcatheter 14 and embolectomy device 10 are then removed from canal 62, as one unit with the retrieval portion 12 and the microcatheter forming a reservoir for containing the clot or emboli 60.

The dimensions and construction of the microcatheter 14, elongated shaft or guide wire 18, intermediate guide wire 20, expander portion 16, retrieval portion 12, legs/spring arms 22, and legs 48 and 52 can vary depending on the size of the canal in which the clot is located, the size and position of the clot, and other factors. The dimensions of the retrieval portion 12 can, for example, be between 0.20 mm to 0.45 mm in diameter when collapsed, and between 0.4 mm to 10 mm when open. In one embodiment the retrieval portion 12 can have a length between 2 mm and 22 mm. The dimensions of the guide wire 18 can in one embodiment be 0.35 mm in diameter, and between 0.20 mm to 0.45 mm in diameter. In another embodiment, the dimensions of the guide wire 18 can be 0.25 mm, and between 0.20 mm to 0.36 mm. The dimensions of the penetrating portion 40 can be 0.25 mm in diameter, or between 0.20 mm and 0.45 mm in diameter. The dimensions of the microcatheter 14 can be an outside diameter (OD) 0.60 mm, and an inside diameter (ID) of 0.43 mm, or with an (OD) between 0.40 mm to 1.37 mm, and an (ID) between 0.25 mm to 0.75 mm. The dimensions of the expander portion 16 can, for example, be between 10 mm and 44 mm in length. The length of the intermediate guide wire 20 can be up to 28 mm. Other dimensions are possible.

The instruments used to position and manipulate the micro catheter 14 and the guide wire 18 can be standard devices or devices specifically designed for use with the invention. Although the legs have been described as spring arms 22, the invention is also useful when the legs/spring arms 22 are moved laterally outward by a force other than a spring force, as where the legs are driven laterally outward by a motor of some kind, such as an osmotic pump. In other embodiments, the mid-portion 30 of the legs/spring arms 22 can be at least partially elastic or can comprise a hinge structure to permit bending of the legs/spring arms.

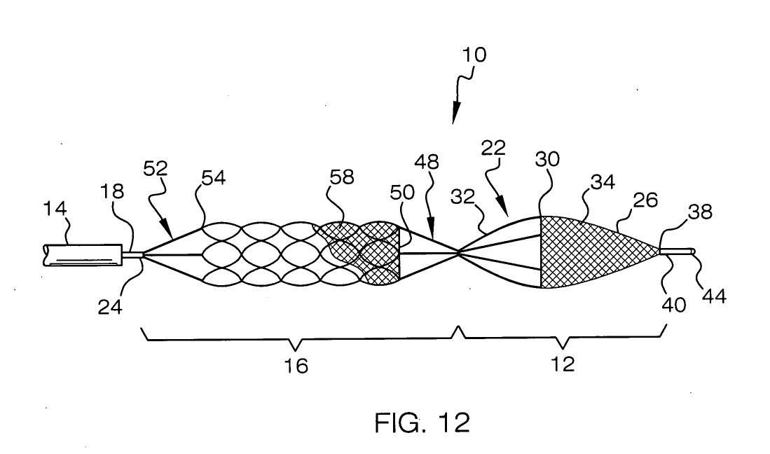

Other embodiments are possible. There is shown in FIG. 12 an embodiment of the embolectomy device 10 where the intermediate connecting wire 20 is absent, and the retrieval portion 12 is connected directly to the expander portion 16. Proximal portions 32 of the spring arms 22 can be connected to the legs 48 at the distal end 50 of the expander portion 16. The remaining aspect can remain as discussed above in the first embodiment shown in FIGS. 1-11.

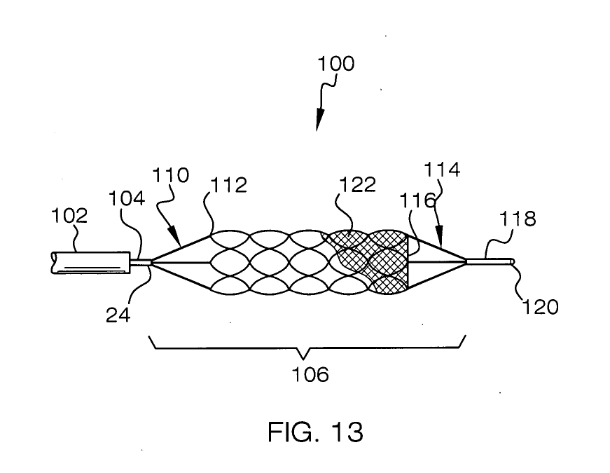

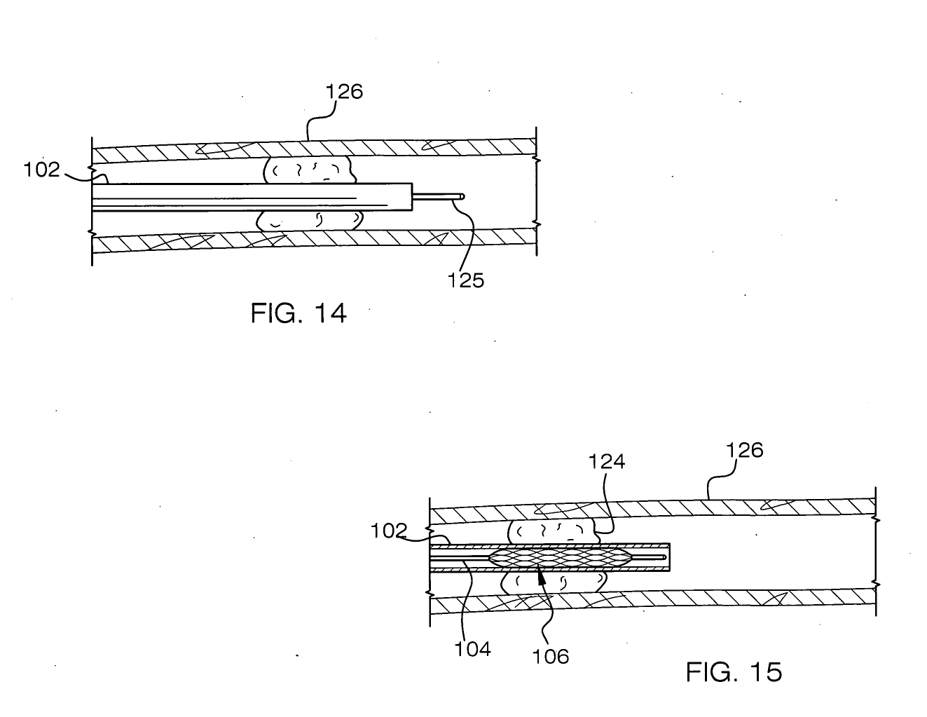

There is shown in FIGS. 13-17 an alternative embodiment of an embolectomy device 100 according to the present invention. In FIG. 13, the embolectomy device 100 is shown fully extended. In FIGS. 14-17, the embolectomy device 100 is shown partially extended in various stages during operation in a body canal. The embolectomy device 100 is positionable in and movable within a catheter 102, such as a microcatheter. The embolectomy device 100 includes an elongated shaft such as proximal wire 104 extending through an opening in the microcatheter 102 and an expander portion 106. The proximal wire 104 can have a proximal end (not shown) and distal end 108. The expander portion 106 can be provided at the distal end 108 of the proximal wire 104.

The expander portion 106 may be formed from or attached to the proximal wire 104. The expander portion 106 is disposed about the long axis A of the embolectomy device 100, and may be baised or otherwise moveable to extend outwardly when not constrained by the microcatheter 102. The expander portion 106 may have a plurality of legs 110 at a proximal end 112 of the expander portion, and a plurality of legs 114 at a distal portion end 116 of the expander portion. The proximal end 112 of expander portion 106 can be connected to the distal end 108 of the proximal wire 104. Legs 110 and 114 may be spring arms, and biased or otherwise moveable to extend outwardly when not constrained by the microcatheter 102. Additionally, the construction and arrangement of spring arms 110 and 114 can be varied. In the embodiment shown, the spring arms 110 and 114 are elongated and substantially radially disposed about a long axis A of the embolectomy device 100. Spring arms 110 can be formed separately and attached to the proximal wire 104. Spring arms 110 can be integral with the proximal wire 104, and can be formed by suitable techniques, such as, but not limited to, computer-controlled laser cutting, plastic injection molding, or casting.. In one embodiment, spring arms 110 and 114 are at least partially made of an elastic material, such as plastic or metal.

The expander portion 106 can be of different designs, but can be a mesh metallic or polymeric device similar to a stent in that it is capable of a first position in which it is tightly compacted laterally, and in another position expands laterally. It is important to understand the expander portion 106 is only stent like, and is not intended to be left in place within a body canal. The expander portion 106 uses this lateral expansion force to spread clot material concentrically outwardly toward the walls of the body canal, opening a lumen through the body canal. In the high energy, compacted state the expander portion 106 is contained within a catheter. When moved out of the catheter, the expander portion 106 assumes an expanded, lower energy state. The expander portion 106 can be completely or partially coated with a polymer, and/or with a therapeutic substance. The expander portion 106 can be porous, non-porous, or partially porous. The expander portion can be formed by suitable techniques, such as, but not limited to, computer-controlled laser cutting, plastic injection molding, or casting.

Expansion of the expander portion 106 is controlled by suitable means, such as the presence of the microcatheter 102. When the microcatheter 102 is removed or pulled back from the expander portion 106, the expander portion is expanded either by a spring force or a suitable motor.

An atraumatic distal portion 118 can be provided at the distal end of the embolectomy device 100 for preventing the embolectomy device from penetrating or piercing a body canal as it is moved therethrough. The distal portion 118 can be part of legs 114 or can be a separate structure to which legs 114 are affixed. The distal portion 118 can be flexible and non-rigid. The distal portion 118 can include a blunt atraumatic tip 120.

A mesh or net 122 can cover a portion or all of the expander portion 106 in order to better engage clot material. The net 112 can be constructed from a porous or a substantially non-porous material, such as a flexible plastic net or solid sheet material, and/or a biocompatible or non-thrombogenic polymer. The net 122 can be integral with the expander portion 106.

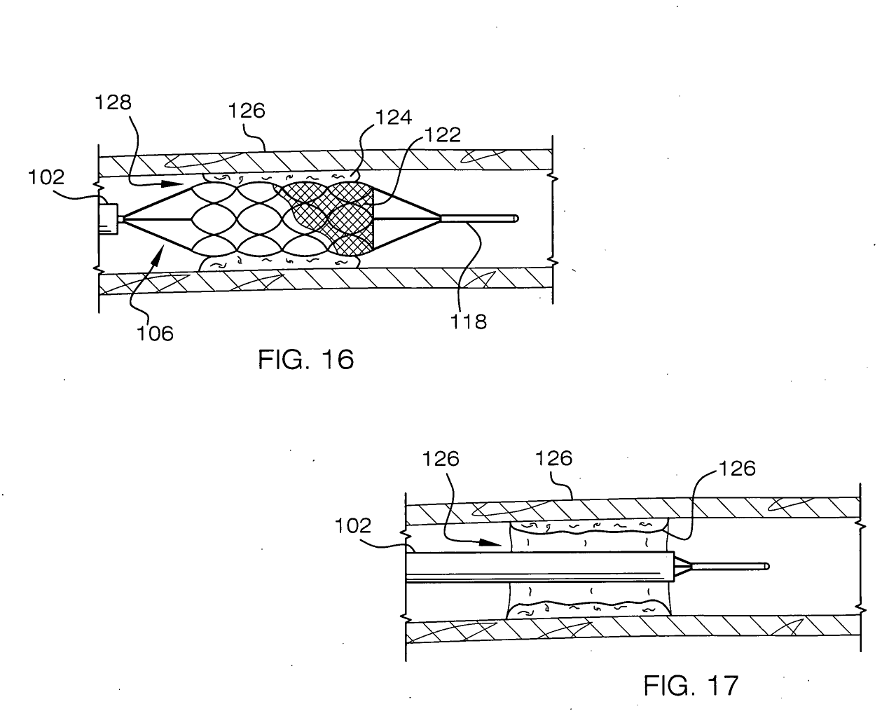

Operation of the embolectomy device 100 is shown in FIGS. 14-17. In FIG. 14, the microcatheter 102 is shown positioned within the body canal 126 through and beyond clot 124 using a steerable microwire 125 and standard interventional radiology techniques. With the microcatheter 14 in position in the body canal 126 and through the clot 124, the steerable microwire is removed from the microcatheter. A contrasting agent can be injected into the microcatheter 102 to confirm the microcatheter is correctly positioned through the clot 124. While maintaining the position of microcatheter 102 the embolectomy device 100 is inserted into the microcatheter to position the expander portion 106 across the clot 124 (FIG. 15). The embolectomy device 100 is extended from the microcatheter 102 by withdrawing the microcatheter 102 proximally while maintaining the positioning of the embolectomy device. Continued withdrawal of the microcatheter 102 exposes the expander portion 106, which is expanded by either spring force or a suitable motor, pressing the clot 124 against the body canal 126 (FIG. 16) and creating a lumen 128. The expander portion 106 can then be retracted into the microcatheter 102 by inserting the microcatheter distally over the expander portion 106. With the expander portion 106 contained within the microcatheter 102, the embolectomy device 100 can be retracted leaving the lumen 128 and at least partially restoring fluid flow through the body canal 126 (FIG. 17).

The dimensions and construction of the microcatheter 102, elongated shaft or guide wire 104, expander portion 106, retrieval portion 12, and legs 110 and 114 can vary depending on the size of the canal in which the clot is located, the size and position of the clot, and other factors. The dimensions of the guide wire 104 can in one embodiment be 0.35 mm in diameter, and between 0.20 mm to 0.45 mm in diameter. In another embodiment, the dimensions of the guide wire 104 can be 0.25 mm, and between 0.20 mm to 0.36 mm. The dimensions of the penetrating portion 118 can be 0.25 mm in diameter, or between 0.20 mm and 0.45 mm in diameter. The dimensions of the microcatheter 102 can be an outside diameter (OD) 0.60 mm, and an inside diameter (ID) of 0.43 mm, or with an (OD) between 0.40 mm to 1.37 mm, and an (ID) between 0.25 mm to 0.75 mm. The dimensions of the expander portion 106 can, for example, be between 10 mm and 44 mm in length. Other dimensions are possible.

A number of embodiments of the present invention have been described. Nevertheless, it will be understood that various modifications may be made without departing from the spirit and scope of the invention. Accordingly, other embodiments are within the scope of the following claims.

What is claimed is:

1. An embolectomy device, comprising:
an elongated shaft positionable in and movable within a catheter, said elongated shaft having a distal end portion;
an intermediate elongated shaft positionable in an movable with the catheter; said intermediate elongated shaft having proximal and distal ends;
an expander portion having proximal and distal ends, said expander portion having a first plurality of legs connecting said proximal end of said expander portion to said distal end portion of said elongated shaft and a second plurality of legs connecting said distal end of said expander portion to said proximal end of said intermediate elongated shaft; and
a retrieval portion having a plurality of legs having proximal and distal portions, said proximal portion being attached to said distal end of said intermediate elongated shaft at said proximal portion of said of plurality of legs of said retrieval portion;
wherein said plurality of legs of said retrieval portion being movable laterally outward from a long axis of the embolectomy device when said retrieval portion has been moved out of the catheter; and
wherein said expander portion being biased to expand laterally outward from said long axis of the embolectomy device when said expander portion has been moved out of the catheter to engage a clot.

2. The embolectomy device of claim 1, further comprising:
an atraumatic distal portion extending distally from said distal end portion of said plurality of legs of said of retrieval portion.

3. The embolectomy device of claim 1, further comprising a retrieval net fixed to said retrieval portion.

4. The embolectomy device of claim 3, wherein said net is constructed from a polymeric material.

5. The embolectomy device of claim 1, wherein said expander portion is at least partially covered by a net.

6. The embolectomy device of claim 5, wherein said net comprises a biocompatible or non-thrombegenic material.

7. The embolectomy device of claim 1, further comprising the catheter.

8. An embolectomy device, comprising:
an elongated shaft positionable in and movable within a catheter, said elongated shaft having a distal end portion;
an intermediate elongated shaft positionable in an movable with the catheter; said intermediate elongated shaft having proximal and distal ends;
an expander portion having proximal and distal ends, said expander portion having a first plurality of legs connecting said proximal end of said expander portion to said distal end portion of said elongated shaft and a second plurality of legs connecting said distal end of said expander portion to said proximal end of said intermediate elongated shaft;
retrieval portion having a plurality of legs having proximal and distal ends and a mid-portion between said proximal and distal ends, said proximal end being attached to said distal end of said intermediate elongated shaft at said proximal end of said of plurality of legs of said retrieval portion;
wherein said mid-portion of said legs being movable laterally outward from a long axis of the embolectomy device when said retrieval portion has been moved out of the catheter; and
wherein said expander portion being biased to expand laterally outward from said long axis of the embolectomy device when said expander portion has been moved out of the catheter to engage a clot.

9. The embolectomy device of claim 8, further comprising a retrieval net fixed to said plurality of legs of said retrieval portion between said mid-portion and said distal end thereof.

10. The embolectomy device of 8, wherein said plurality of legs of said retrieval portion are spring arms.

11. The embolectomy device of claim 10, wherein said spring arms comprise a bend at said mid-portion defining proximal and distal portions, said bend biasing said spring arms to a lateral outward position.

12. The embolectomy device of claim 8, further comprising:
a atraumatic distal portion extending distally from said distal end portion of said plurality of legs of said of retrieval portion.

13. The embolectomy device of claim 8, wherein said expander portion is at least partially covered by a net.

14. The embolectomy device of claim 13, wherein said net comprises a biocompatible or non-thrombegenic material.

15. The embolectomy device of claim 8, further comprising the catheter.

16. The embolectomy device of claim 11, wherein said spring arms are integral with said intermediate elongated shaft.

17. The embolectomy device of claim 11, wherein said spring arms are attached to said intermediate elongated shaft.

18. An embolectomy device, comprising:
an elongated shaft positionable in and movable within a catheter, said elongated shaft having a distal end portion;
an expander portion having proximal and distal ends, said expander portion having a first plurality of legs connecting said proximal end of said expander portion to said distal end portion of said elongated shaft and a second plurality of legs connecting said distal end of said expander portion to said proximal end of said intermediate elongated shaft; and
wherein said expander portion being biased to expand laterally outward from said long axis of the embolectomy device when said expander portion has been moved out of the catheter to engage a clot.

19. The embolectomy device of claim 18, further comprising:
a atraumatic distal portion extending distally from said distal end of said expander portion.

20. The embolectomy device of claim 18, wherein said expander portion is at least partially covered by a net.

21. The embolectomy device of claim 20, wherein said net comprises a biocompatible or non-thrombegenic material.

22. The embolectomy device of claim 18, further comprising the catheter.

23. A method of performing an embolectomy, comprising the steps of:
providing an embolectomy device comprising an elongated shaft positionable in and movable within a catheter, said elongated shaft having a distal end portion, an intermediate elongated shaft positionable in an movable with the catheter, said intermediate elongated shaft having proximal and distal ends, an expander portion having proximal and distal ends, said expander portion having a first plurality of legs connecting said proximal end of said expander portion to said distal end portion of said elongated shaft and a second plurality of legs connecting said distal end of said expander portion to said proximal end of said intermediate elongated shaft, a retrieval portion having a plurality of legs having proximal and distal portions, said proximal portion being attached to said distal end of said intermediate elongated shaft at said proximal portion of said of plurality of legs of said retrieval portion, said plurality of legs of said retrieval portion being movable laterally outward from a long axis of said elongated shaft when said retrieval portion has been moved out of the catheter, said expander portion being biased to expand laterally outward from said long axis of the embolectomy device when said expander portion has been moved out of the catheter to engage an occlusion, and a retrieval net fixed to said retrieval portion;
manipulating the embolectomy device within a catheter to position the expander portion across an occlusion in a body canal;
expanding the expander portion to move the occlusion toward said body canal, thereby creating a lumen;
retracting the expander portion;
moving the retrieval portion proximally so as to engage said occlusion with the retrieval net; and
removing the catheter and embolectomy device as one from the body canal with portions of the occlusion contained by the catheter and the retrieval portion of the embolectomy device.

24. A method of performing an embolectomy, comprising the steps of:
providing an embolectomy device comprising an elongated shaft positionable in and movable within a catheter, said elongated shaft having a distal end portion, an expander portion having proximal and distal ends, said expander portion having a first plurality of legs connecting said proximal end of said expander portion to said distal end portion of said elongated shaft and a second plurality of legs connecting said distal end of said expander portion to said proximal end of said intermediate elongated shaft, said expander portion being biased to expand laterally outward from said long axis of the embolectomy device when said expander portion has been moved out of the catheter to engage an occlusion;
manipulating the embolectomy device within a catheter to position the expander portion across an occlusion in a body canal;
expanding the expander portion to move the occlusion toward said body canal, thereby creating a lumen;
retracting the expander portion; and
removing the catheter and expander portion from the body canal leaving the lumen open.
